(19) United States
(12) Patent Application Publication
(10) Pub. No.: US 2020/0384153 A1
(43) Pub. Date: Dec. 10, 2020

(54) MULTI-LAYER HAEMOSTAT PATCH COMPRISING BETA-CHITIN

(71) Applicants: Peter-John WORMALD, North Adelaide (AU); Stephen Carl MORATTI, Dunedin (NZ); Lyall Robert HANTON, Dunedin (NZ); Simon ROBINSON, Wellington (NZ); Alistair JUKES, Adelaide (AU)

(72) Inventors: Peter-John WORMALD, North Adelaide (AU); Stephen Carl MORATTI, Dunedin (NZ); Lyall Robert HANTON, Dunedin (NZ); Simon ROBINSON, Wellington (NZ); Alistair JUKES, Adelaide (AU)

(21) Appl. No.: 16/905,349

(22) Filed: Jun. 18, 2020

Related U.S. Application Data
(63) Continuation-in-part of application No. PCT/AU2018/000267, filed on Dec. 21, 2018.

(30) Foreign Application Priority Data
Dec. 21, 2017 (AU) 2017905127

(51) Int. Cl. A61L 27/20; A61L 27/36

(52) U.S. Cl. CPC A61L 27/20; A61L 27/3641

(57) ABSTRACT

A haemostat agent in the form of a multi-layer patch comprising at least first and second layers, wherein said first layer comprises a composition comprising fibrous β-chitin (which may be sourced from a marine source such as squid pens) and one or more biocompatible polymer (for example, a polyethylene glycol (PEG) polymer or sodium polyphosphate (PolyP)), and said second layer comprises a film layer comprising fibrous β-chitin. In one particular application, the haemostat agent is used for treating carotid artery injury associated with endonasal surgery.

### CROSS-REFERENCE TO RELATED APPLICATIONS

This application is a continuation-in-part of International Application No. PCT/AU2018/000267 filed 21 Dec. 2018, which claimed the benefit of Australia Patent Application No. 2017905127 filed 21 Dec. 2017, the entire contents of each of which are incorporated herein by reference in their entirety.

### TECHNICAL FIELD

The present disclosure relates to a novel haemostat agent comprising the polysaccharide, β-chitin. In one particular application, the novel haemostat agent is used for treating carotid artery injury associated with endonasal surgery.

### BACKGROUND

Bleeding during surgery is inevitable and significant time and effort is put in by the surgical team to control such haemorrhage. In most circumstances, the surgical approach creates access to the bleeding vessel and this allows the surgeon to control the haemorrhage with traditional methods such as compression, suturing or tying the vessel. However, there are circumstances, especially with endoscopic surgery, where access is limited and the ability to manage such haemorrhage with traditional methods is impossible. An example of such a situation is the extended endonasal approach to the skull base. This surgical approach continues to gain popularity and increasing numbers of pathologies are now removed or treated using this approach. As more surgeons become proficient at these techniques and extend their skills, it is important to acknowledge that there is a real risk of potentially catastrophic major arterial haemorrhage (Vaz-Guimaraes et al., 2015; Gardner et al., 2013; Padhye et al., 2014). This is typically from the cavernous segment of the carotid artery but, as approaches to deeper and more complex pathologies are developed, the risk to the anterior communicating artery complex and the basilar regions will also increase. Using large, pooled case series, has put this risk at between 1-9% in extended endonasal approaches (Valentine et al., 2011; Valentine and Wormald, 2011).

Unfortunately, bleeding at the skull base is also often difficult to control as anatomic restrictions can preclude the application of surgical clips such as vessel clip closures and other devices. Currently, the gold standard for treatment of these injuries is the application of patches of crushed muscle taken from the patient (“muscle patches”). However, whilst this treatment is able to control the bleeding, there is a high incidence of pseudoaneurysm development over the post-operative period. Indeed, some estimates on the incidence of the development of a post-operative pseudoaneurysm range as high as 66%, but most pooled series put this in the region of 20-40% (Gardner et al., 2013; Berker et al., 2010; Biswas et al., 2009). In addition, treatment of the bleeding with muscle patches requires a second surgeon to harvest the muscle and some considerable time to prepare and then apply, which can mean that significant blood loss can occur whilst gaining definitive haemostasis. Accordingly, alternatives to the use of surgical clips and muscle patches are required in the skull base surgeon's armamentarium when dealing with potentially catastrophic arterial bleeding.

As mentioned above, the present disclosure relates to a novel haemostat agent. In work leading to this novel haemostat agent, an ovine endoscopic carotid artery injury model was developed which can be used for both research and surgical training (Valentine and Wormald, 2011). Several techniques have been trialled on this model including the use of a muscle patch and a vessel clip closure (via an AnastoClip device; LeMaitre Vascular, Inc., Burlington, Mass., United States of America). However, in an effort to identify alternatives that may reduce the time to haemostasis, blood volume loss, and/or the incidence of pseudoaneurysm formation, three commercially available haemostat agents (two in combination) were trialled in the model along with the novel haemostat agent which comprises a patch comprised of β-chitin that may be preferably derived from squid pen (otherwise known as the gladius, which is the vestigial internal shell of the squid).

### SUMMARY

The present disclosure provides, in a first aspect, a novel haemostat agent in the form of a multi-layer patch comprising at least first and second layers, wherein said first layer comprises a composition comprising fibrous β-chitin and one or more biocompatible polymer, and said second layer comprises a film layer comprising fibrous β-chitin.

In a second aspect, the present disclosure provides a method of promoting haemostasis and/or wound healing in a human subject, said method comprising applying a haemostat agent according to the first aspect to a desired site such as a bleeding blood vessel, injury or other wound where bleeding management is desirable.

In a third aspect, the present disclosure provides a method of producing a haemostat agent according to the first aspect, comprising the steps of:
(i) forming a film of fibrous β-chitin, preferably wherein the fibrous β-chitin comprises fibres having a typical length in the range of about 1 μm to 150 μm,
(ii) forming a suspension of fibrous β-chitin, preferably wherein the fibrous β-chitin comprises fibres having a typical length in the range of about 1 μm to 150 μm, and one or more biocompatible polymer (preferably polyethylene glycol (PEG) and/or sodium polyphosphate);
(iii) applying the suspension of step (ii) to one side of the film of step (i) to produce a layered intermediate product, and
(iv) thereafter freeze-drying the intermediate product such that a foamed composition of the β-chitin fibres and biocompatible polymer is formed and provides a first layer that is bonded or laminated to the film which forms a second layer.

### BRIEF DESCRIPTION OF FIGURES

FIG. 1 provides an image of a haemostat agent (patch) according to the present disclosure;

FIG. 2 provides an SEM microscopy image (×15000) of a haemostat agent according to the present disclosure, following sterilisation through application of gamma irradiation;

FIG. 3 provides an image of a comparator haemostat agent, comprising a first layer composed only of β-chitin (ie no biocompatible polymer), showing delamination of the layers;

FIG. 4 provides graphical results showing the effect of various β-chitin patches on platelet aggregation, as measured by a lactate dehydrogenase enzyme (LDH) assay; the OD value of β-chitin (Chitin), Chi/F127, Chi/PEO, Chi/5% Ca, Chi/10% Ca, Chi/20% Ca, Chi/Thick, and Chi/PP patches as well as a NC (negative control) were normalised to a PC (positive control) (*p<0.05 and **p<0.001, compared to chitin, analysed by Kruskal-Wallis one-way ANOVA with Dunn's test, n=5);

FIG. 5 provides graphical results showing the effect of various β-chitin patches on thrombin generation, as measured by a thrombin-antithrombin assay (ng/mL) (*p<0.05 and **p<0.001, compared to Chi/PP, analysed by Kruskal-Wallis one-way ANOVA with Dunn's test, n=5);

FIGS. 6A & 6B graphically shows the cytotoxic effects of various β-chitin patches, Surgicel® and FloSeal haemostats, as measured by alamar blue: Alamar blue assay at (A) 24-hours and (B) 48-hours post-treatment, showing the cell viability with β-chitin (Chitin), Chi/F127, Chi/PEG, Chi/5% Ca, Chi/10% Ca, Chi/20% Ca, Chi/Thick, Chi/PP, Surgicel®, FloSeal and PC (positive control) treatments, normalised to NC (negative control) (*p<0.05 and **p<0.001, compared to Chitin, analysed by Kruskal-Wallis one-way ANOVA with Dunn's test, n=5); and

FIGS. 7A & 7B provide graphical results showing the effects of gauze, various β-chitin patches, Surgicel® and FloSeal haemostats on: (A) the mean bleeding time and (B) the mean blood loss, in a rat femoral arterial injury experiment (*p<0.05 and **p<0.001, compared to chitin, analysed by Kruskal-Wallis one-way ANOVA with Dunn's test).

### DETAILED DESCRIPTION

Beta((3)-chitin is a form of poly-N-acetyl glucosamine (pGlcNAc) comprising N-acetyl-D-glucosamine (more particularly, 2-(acetylamino)-2-deoxy-D-glucose) monomers linked by covalent β-(1→4)-linkages. This carbohydrate polymer (polysaccharide) can be derived from animal, fungal and algal sources, where it forms a major component of, for example, crustacean exoskeletons, squid pens (gladii), the wings of insects, marine algae and fungal mycelial mats. In its natural state, β-chitin exists in a crystalline form wherein the pGlcNAc polymer molecules are arranged in parallel lines (cf α-chitin where the polymer molecules are arranged in antiparallel lines). This material is capable of being formed into nanofibres measuring, for example, 1-150 micrometres (μm) in length and 20-80 nanometres (nm) in diameter.

It has been known for some time that chitin possesses some interesting properties and activities. In particular, properties useful for achieving haemostasis were described in relation to gastric varices in 1998 (Kulling et al., 1998). In vitro studies investigating these properties have shown that both α and (β-chitin can increase red blood cell aggregation and endothelial-dependant vasoconstriction and, additionally, are also able to activate platelets (see, for example, Okamoto et al., 2003; Agrawal et al., 2014; Whang et al., 2005; Kadokawa et al., 2011). Cognisant of these activities, a three-step mechanism by which chitin, and particularly β-chitin, may achieve haemostasis has been proposed (Thatte et al., 2004): (1) pGlcNAc binds to immobilised plasma proteins such as fibrinogen and/or platelets directly bind to pGlcNAc; (2) Integrin-mediated platelet activation then activates the intrinsic coagulation pathway via Hageman activation factor (Factor XII) to generate thrombin and the formation of a stable clot (which may be further stabilised by the tendency for platelets to aggregate on pGlcNAc matrices and generate vasospastic substances such as thromboxane and serotonin); and (3) Clot retraction via platelet mediators and local vasospasm accelerates wound healing.

The present disclosure relates to the use of this ability of chitin to achieve haemostasis by including β-chitin as an essential integer of a novel haemostat agent.

In particular, the present disclosure provides, in a first aspect, a novel haemostat agent in the form of a multi-layer patch comprising at least first and second layers, wherein said first layer comprises a composition comprising fibrous β-chitin and one or more biocompatible polymer, and said second layer comprises a film layer comprising fibrous β-chitin.

Preferably, β-chitin is the only haemostatic carbohydrate polymer material present in the haemostat agent.

The haemostat agent may take the form of a patch in any suitable shape and with any suitable dimension (eg thickness) to readily enable application to a site where haemostasis is desired. Typically, the agent may be a square- or rectangular-shaped patch with dimensions of, for example, 3 cm×3 cm, 4 cm×4 cm, 5 cm×3 cm or 10 cm×5 cm, or otherwise the agent may be circular in shape (eg with a diameter of, for example, about 1 cm to about 5 cm or, preferably, about 2 cm or about 3 cm). Preferably, the thickness of the patch will ensure flexibility and may be, for example, 0.25 cm to 1.5 cm thick or, more preferably, 0.45 cm to 1.0 cm thick. In some preferred embodiments, the patch may have a thickness of about 0.45 cm, about 0.5 cm, about 0.6 cm, about 0.65 cm, about 0.7 cm, about 0.75 cm or about 0.8 cm.

In use, the patch will be applied so that the first layer inwardly “faces” the desired site (eg the first layer faces the bleeding blood vessel or injury) to at least substantially provide the haemostatic activity or effect of the patch. As such, the second layer may be regarded as a “backing layer” and may provide, for example, mechanical strength or reinforcement. However, it is to be understood that the patch may be provided with one or more additional layers which may be outwardly facing (ie the one or more additional layer may be on the outside of the second layer; that is, on the opposite side to the first layer), and as such, may be for example, a medical-grade foam backing sheet, and/or may be intermediate to the first and second layers (ie one or more layer sandwiched between the first and second layers). The first and second layers (and, optionally, any additional layers) may be bonded or laminated together by any of the means or techniques well known to those skilled in the art (including, for example, bonding with an adhesive (eg spot bonding), preferably a biocompatible adhesive such as fibrin glue, and chemical treatment to achieve cross-linking between elements of the layers (eg cross-linking between β-chitin molecules of the bonded layers)). In addition, it has been found that by contacting the first and second layers during drying, the layers may become sufficiently physically bonded (possibly through some interweaving of the β-chitin molecules on the respective surfaces of the first and second layers).

The first layer comprises a composition comprising fibrous β-chitin and one or more biocompatible polymer. Preferably, the composition has a foamed (or sponge-like) structure or is otherwise comprised of a structure providing a random arrangement or network (preferably interconnecting) of voids and/or channels which may, for example, allow for the passage of blood into the structure which may assist in achieving haemostasis. As such, the structure may also contribute to a level of “softness” and/or flexibility in the patch to assist in the ready application to the desired site. To achieve a foamed structure, a suspension of fibrous β-chitin and the one or more biocompatible polymer may be prepared and subjected to, for example, a routine technique such as freeze drying (ie lyophilisation) or supercritical drying. Thus, in some embodiments, the first layer comprises a freeze dried composition comprising fibrous β-chitin and one or more biocompatible polymer.

The β-chitin of the first layer is provided in a fibrous form. For example, the β-chitin is present as fibres (or “nanofibres”), wherein typically the length of the fibres is in the range of about 1 μm to about 500 μm or, more preferably about 1 μm to about 150 μm (eg about 25 μm to about 75 μm, or 60 μm to 100 μm or 75 μm to 150 μm), and have a diameter of between about 10 nm and 100 nm (more preferably, 20 nm to 80 nm). The β-chitin fibres may preferably comprise a parallel arrangement of the constituent β-chitin molecules, which is considered to be advantageous inasmuch as it is believed that the parallel β-chitin molecules may act to “trap” platelets at the site where they can then exert their activity in forming a stable clot. Preferably, the β-chitin is obtained from a natural source such as a marine source such as crustacean exoskeletons or marine algae, but more preferably, squid pens (eg from the New Zealand arrow squid (Nototodarus sloanii)). However, synthetic β-chitin may also be used. The β-chitin can be readily prepared from natural sources by, for example, cutting the material into small pieces, drying the material pieces and thereafter grinding the dried material into a powder (eg comprising β-chitin fibres) which may pass through, for example, a 0.420 mm mesh sieve. However, other preparation processes will be readily apparent to those skilled in the art.

The β-chitin fibres of the first layer may be coated, impregnated and/or suspended in the one or more biocompatible polymer. As such, the biocompatible polymer(s) may provide the composition with improved durability by, for example, negating possible brittleness of the β-chitin fibres. The one or more biocompatible polymer may be present in the composition of the first layer in an amount of about 2 to about 50 w/w (based on the amount of β-chitin present), more preferably in an amount in the range of about 10 to about 40% w/w, and most preferably, in an amount of about 15 to about 25% w/w. In some preferred embodiments, the amount of the one or more biocompatible polymer present in the composition is about 20% w/w (based on the amount of β-chitin present).

The biocompatible polymer may be selected from any of the organic or inorganic polymer types applicable to the medical field (eg as used in surgical tools and consumables such as swabs, patches and the like, or medical devices) including, for example, synthetic and naturally occurring polymers including those which may also be biodegradable. Suitable inorganic polymers include polyphosphates (eg sodium polyphosphates), polysiloxanes and polyphosphazenes. Suitable organic polymers include synthetic biocompatible polymers such as poly(glycolic acid), poly(lactic acid), poly(e-caprolactone), poly(beta-hydroxybutyric acid), polyethylene terephthalate (PET), polyethylene glycol (PEG), poly(malic acid), poly(tartronic acid) and the copolymers of the monomers used to synthesise these polymers. Other suitable organic polymers include naturally occurring biocompatible polymers such as alginate, chitosan, starch, dextran and albumen. However, in some embodiments, the biocompatible polymer is a polymer other than alginate and/or other than chitosan. Moreover, in some embodiments, the first layer does not comprise gelatin and/or collagen.

In some preferred embodiments, the one or more biocompatible polymer included in the composition is/are selected from the class of polyethylene glycol (PEG) polymers. For example, PEG compounds having a molecular weight in the range of about 0.5 kDa to about 5 kDa or, more preferably, in the range of about 1 kDa to about 2 kDa. In some particularly preferred embodiments, the composition comprises a PEG compound with a molecular weight of about 1 kDa as the biocompatible polymer.

In other preferred embodiments, the one or more biocompatible polymer included in the composition is/are selected from polyphosphates such as sodium polyphosphates (Ray NH, 1979), which may act as a clotting agent. One particularly preferred example is Graham's salt (sodium polyphosphate or “PolyP”). The polyphosphates may optionally be provided as a functionalised surface on biocompatible and/or biodegradable nanoparticles. PolyP is known to modulate blood coagulation by binding coagulation enzymes including kallikrein, thrombin and factor XIa, as well as having various activating roles on certain blood clotting enzymes such as factor XII (Travers et al., 2015).

The composition of the first layer of the haemostat agent may further comprise one or more other substance such as, for example, a filler substance such as a suitable clay or other inert material, keratin, collagen, or a substance conferring some other beneficial effect such as local pain relief (eg lidocaine), or a substance that may contribute to, or enhance, the haemostat effect (eg certain carbohydrate substances such as oxidised cellulose (although, as indicated above, preferably β-chitin is the only haemostatic carbohydrate polymer material present in the haemostat agent), a proteinaceous substance such as thrombin, fibrin or gelatin, which can assist by absorbing surrounding fluid at the site, or calcium (eg in the form of calcium acetate)). Typically, the any one or more other substance would be present in an amount totalling not more than about 10 to about 20% w/w (based on the amount of β-chitin present), but preferably less than about 5% (w/w).

The thickness of the first layer may be in the range of, for example, 0.05 cm to 1.45 cm or, more preferably, 0.1 cm to 0.95 cm. In some preferred embodiments, the first layer thickness may be about 0.45 cm to about 0.65 cm.

The second layer comprises fibrous β-chitin and, as mentioned above, may be regarded as a “backing layer” which may, for example, provide mechanical strength or reinforcement to the haemostat agent. The β-chitin of the second layer may also contribute to the haemostatic effect of the haemostat agent. In addition, the second layer may possess properties that assist or improve the handling of the haemostat agent, particularly when applying the haemostat agent to the desired site. For example, the backing layer may be “non-sticking”, so that once the haemostat agent is applied, it is not “removed” or shifted by sticking to the surgeon's hand or a surgical tool. In addition, the second layer may be blood impermeable to assist in containing blood and other fluid at the site of the bleeding blood vessel or injury.

Like the β-chitin of the first layer, the β-chitin of the second layer is provided in a fibrous form (eg as fibres wherein typically the length of the fibres is in the range of about 1 μm to about 500 μm, about 25 μm to about 400 μm, about 25 μm to about 150 μm or about 1 μm to about 150 μm or, more preferably, in the range of 25 μm to 75 μm, 60 μm to 100 μm or 75 μm to 150 μm, and having a diameter of between about 10 nm and 100 nm, but more preferably, 20 nm to 80 nm). The β-chitin fibres may preferably comprise a parallel arrangement of the constituent β-chitin molecules, and may be obtained from a natural source such as those sources mentioned above or may otherwise comprise synthetic (3-chitin. The β-chitin for use in the second layer is preferably prepared from natural sources by the same process as that which may be used for the β-chitin of the first layer.

While preferably the second layer comprises β-chitin substantially alone, in some embodiments, the second layer may further comprise an organic or inorganic biocompatible polymer. In such embodiments, the biocompatible polymer may be selected from synthetic and naturally occurring polymers including those which may also be biodegradable. Preferably, the biocompatible polymer for use in the second layer will be an organic polymer. Suitable organic polymers include synthetic biocompatible polymers such as poly(glycolic acid), poly(lactic acid), poly(e-caprolactone), poly(beta-hydroxybutyric acid), polyethylene terephthalate (PET), polyethylene glycol (PEG), poly(malic acid), poly(tartronic acid) and the copolymers of the monomers used to synthesise these polymers. Other suitable organic polymers include naturally occurring biocompatible polymers such as alginate, chitosan, starch, dextran and albumen. Other substances such as other carbohydrate substances (eg oxidised cellulose) and proteinaceous substances including fibrin, gelatin, keratin and collagen may also be included. However, in some embodiments, where a biocompatible polymer or other substance is included in the second layer, the biocompatible layer or other substance is other than alginate, other than chitosan, other than gelatin and/or other than collagen. Also, where a biocompatible polymer and/or other substance is included in the second layer, typically the amount of such will total no more than about 20% w/w (based on the amount of β-chitin present), and preferably, less than about 10% or 5% w/w.

The thickness of the second layer will be typical of single layer films and, as such, may be in the range of, for example, 0.02 mm (ie 20 μm) to 0.1 cm. In some preferred embodiments, the second layer thickness may be in the range of 0.025 mm (ie 25 μm) to 0.1 mm (ie 100 μm). Further, in some particularly preferred embodiments, the second layer thickness may be about 0.03 mm (ie 30 μm) or about 0.05 mm (ie 50 μm).

In some particularly preferred embodiments, the haemostat agent will be in the form of a bi-layer patch comprising first and second layers, wherein;

said first layer comprises a foamed composition comprising fibrous β-chitin (preferably sourced from squid pens) and polyethylene glycol (preferably having a molecular weight of about 1 kDa to 2 kDa), wherein said polyethylene glycol is present in an amount of about 20% w/w based on the amount of β-chitin, and

said second layer comprises a film layer comprising fibrous β-chitin.

In some other particularly preferred embodiments, the haemostat agent will be in the form of a bi-layer patch comprising first and second layers, wherein;

said first layer comprises a foamed composition comprising fibrous β-chitin (preferably sourced from squid pens) and sodium polyphosphate, wherein said sodium polyphosphate is present in an amount of about 20% w/w based on the amount of β-chitin, and

said second layer comprises a film layer comprising fibrous β-chitin.

The haemostat agent of the present disclosure is for human use only. In this context, the haemostat agent may provide a fast-acting, field ready or “ready to use” patch for treating a patient at the at the site of a bleeding blood vessel (particularly, a major vessel bleeding), injury or other wound where bleeding management is desirable such as vascular access sites (eg for obtaining and maintaining haemostasis following femoral vascular catheterisation), percutaneous catheters or tubes and sites of surgical debridement. To assist in having the haemostat agent ready to use, individual patches are preferably provided in typical sterile packaging types used in the medical and surgical fields, such as single sterile foil type packages and multi blister type packages. Once removed from its packaging, the patch may be applied to the site and held in place with firm pressure until the bleeding has been controlled, after which the patch may be slowly released. Subsequently, the site may be assessed and the patch left in place or otherwise replaced with a fresh patch, then optionally covered with a further dressing (eg a transparent dressing or a further haemostat patch) in accordance with typical procedures. In the case of major vessel bleeding (eg bleeding associated with carotid injury), those skilled in the art will appreciate that prior to the application of a patch according to the present disclosure, occlusive pressure should preferably be applied to the vessel at a position proximal to the vessel puncture or tear and the pressure maintained until the patch has been applied and haemostasis achieved. Further, those skilled in the art will appreciate that in the case of major vessel bleeding, where the bleeding is “high pressure bleeding”, the haemostat agent may be used in conjunction with a biocompatible adhesive or sealant (eg a bioglue such as fibrin glue, albumin-glutaraldehyde or a cyanoacrylate-based tissue adhesive such as octyl-cyanoacrylate).

In a second aspect, the present disclosure provides a method of promoting haemostasis and/or wound healing in a human subject, said method comprising applying a haemostat agent according to the first aspect to a desired site such as a bleeding blood vessel, injury or other wound where bleeding management is desirable.

The method may be used for, for example, promoting haemostasis and/or wound healing at a vascular access site (eg for obtaining and maintaining haemostasis following femoral vascular catheterisation), wound associated with a percutaneous catheter or tube, site of surgical debridement, or injury. However, of greater significance, the method may be used for promoting haemostasis and/or wound healing at the site of a bleeding blood vessel. In particular, the method may be used in relation to major vessel bleeding (eg bleeding associated with carotid injury, particularly carotid artery injury associated with endonasal surgery).

In a third aspect, the present disclosure provides a method of producing a haemostat agent according to the first aspect, comprising the steps of:
(i) forming a film of fibrous β-chitin, preferably wherein the fibrous β-chitin comprises fibres having a typical length in the range of about 1 μm to 150 μm,
(ii) forming a suspension of fibrous β-chitin, preferably wherein the fibrous β-chitin comprises fibres have a typical length in the range of about 1 μm to 150 μm, and one or more biocompatible polymer (preferably polyethylene glycol (PEG) and/or sodium polyphosphate);
(iii) applying the suspension of step (ii) to one side of the film of step (i) to produce a layered intermediate product, and
(iv) thereafter freeze-drying the intermediate product such that a foamed composition of the β-chitin fibres and the biocompatible polymer is formed and provides a first layer (wherein, in embodiments including one or more biocompatible polymer, the said β-chitin fibres are coated, impregnated and/or suspended in the biocompatible polymer) that is bonded or laminated to the film which forms a second layer.

The first and second layers may become sufficiently physically bonded simply by virtue of being in contact during the freeze-drying step (iv), possibly through some interweaving of the β-chitin fibres at the respective surfaces of the first and second layers.

The haemostat agent of the present disclosure is hereinafter further described by way of the following non-limiting example(s) and accompanying figures.

### EXAMPLE(S)

### Example 1

### Methods and Materials

Animal Model

Eighteen (18) merino sheep underwent a general anaesthetic using intravenous (iv) ketamine and diazepam for induction and intubated. Isoflurane was given for maintenance of anaesthesia and the animals placed supine with neck extended. Intramuscular (im) antibiotics were given. A midline neck dissection was performed and an arterial line and 12-french central line placed in the left common carotid and internal jugular vein respectively. The right common carotid was then dissected free from surrounding strap muscles and adventitia and encased in a modified S.I.M.O.N.T endoscopic trainer (Pro-delphus, Sao Paulo, Brazil) following a previously described procedure (Valentine and Wormald, 2011). A curved aneurysm clip was placed half way across the common carotid endoscopically to isolate a segment of the vessel. A scalpel was then used to make a standardised 4 mm linear incision in the carotid artery. Warmed intravenous saline (Baxter Australia, Old Toongabbie, NSW, Australia) was started through the central line. The aneurysm clip was then removed and the vessel allowed to bleed for 5 seconds. Haemorrhage control was then achieved by endoscopic application of the haemostat agents being tested. A two surgeon, four hand technique was used. Once bleeding had been controlled, the area was observed for 10 minutes to ensure no re-bleeding. The endoscopic training model was removed, the central and arterial lines removed, and the neck incision sutured closed in a single absorbable layer. The animals were then given im analgesia, recovered and observed with monitoring of vital signs, feeding and behaviour. Subsequently, the animals were kept alive for 3-months post operatively and then underwent magnetic resonance angiography (MRA) of the neck vessels to determine if a pseudoaneurysm had formed. Animals were then humanely euthanised and the right sided carotid removed en-bloc with surrounding strap muscles. In two sheep from each group, this carotid was placed in electron microscopy fixative for 7 days and in four sheep from each group, this carotid underwent 7 days of formalin fixation. The specimens were then prepared for formal histology and electron microscopy.

Haemostat Agents

The 18 merino sheep were divided into 3 groups. 6 animals underwent the application of a Tachosil® patch (Baxter Healthcare Corporation, Deerfield, Ill., United States of America), another 6 animals underwent the application of Evicel® fibrin sealant and a Surgicel SNoW™ absorbable haemostat (Ethicon US LLC, Sommerville, N.J., United States of America) and 6 received a β-chitin haemostat patch according to the present disclosure prepared as described in paragraph [0040] below.

Tachosil® is a topical sealant patch (9.5×4.8 cm) that consists of human fibrinogen (3.6 to 7.4 mg (5.5 mg) per cm² and human thrombin (1.3 to 2.7 Units (2.0 U) per cm²) (Matonick and Hammond, 2014) coated onto an equine-derived collagen sponge. It is thought to function through activation of fibrinogen into fibrin monomers with subsequent polymerisation. The formation of fibrin polymers (ie a fibrin clot), and platelet activation, then leads to the activation of the coagulation cascade with further adherence to the wound surface via further thrombin-mediated fibrin polymerisation and conglutination of the patch's collagen matrix and the wound surface, forming a tight seal (Hickerson et al., 2011). Tachosil® is indicated for use in cardiac and hepatic surgery as an adjunct to haemostasis where it is placed as a sheet upon the bleeding surface (Matonick and Hammond, supra).

Evicel®/SNoW™ consists of 55-85 mg/ml fibrinogen and 800-1200 IU/ml human thrombin in frozen solution provided in separate vials which are mixed in a sterile applicator. Using the applicator, the solution is applied by dripping or spraying onto the wound. It is believed to function in a similar manner to the Tachosil® patch; that is, by the thrombin-activated conversion of the fibrinogen into fibrin monomers for subsequent polymerisation to form a fibrin clot. Evicel® has been trialled in, inter alia, endoscopic endonasal surgery using post-operative mucosal bleeding rates as an outcome measure (Vaiman, 2002; Vaiman, 2005). Post-operative bleeding occurred in 0-5% of patients in the Evicel® groups compared with 23-37% of patients in a group treated by nasal packing alone (Vaiman, 2005).

Preparation of β-chitin Haemostat Agent (Patch)

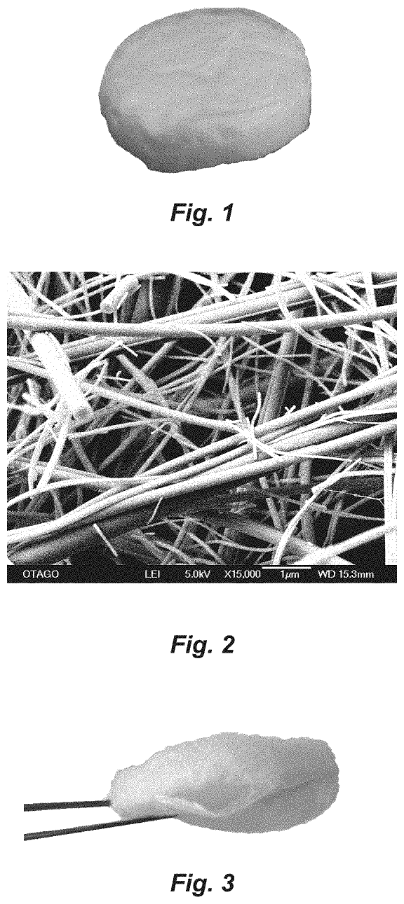

Squid pens obtained from the New Zealand arrow squid (Nototodarus sloanii) were cut into small pieces, washed with ethanol to dry, and ground to a fine powder in a coffee grinder. The powder (7.50 g) was sieved through a sieve with a 0.420 mm mesh size, and stirred in NaOH (1 M, 200 mL) for 3 days. The solid was then filtered off, washed with H₂O until the pH was neutral, washed with ethanol, and air dried on the funnel to give a sticky solid which eventually (30 min) dried to a white solid (2.53 g, 33%). A portion (1.00 g) was then sonicated with acetic acid (1% v/v, 200 mL) with a probe sonicator (UP100H; Hielscher Ultrasonics GmbH, Teltow, Germany) for 30 min, frequently alternating the site in which the probe was “seated”, to give an opaque thick suspension with no separate liquid. To determine the concentration of chitin fibres in suspension in this liquid, a portion (11.6 g) was filtered on a Buchner flask and the collected solid then dried at 45° C. O/N. This gave a solid film of chitin fibres (56 mg). To make a bulk suspension containing polyethylene glycol (PEG) at 20% w/w PEG (based on chitin content), melted PEG (1 kDa, 168 mg; Sigma-Aldrich; St Louis, Mo., United States of America) was dissolved in a portion of the remaining solution (139.2 g). Backing films were then made by filtering the chitin suspension (without PEG) (11.6 g) using a Buchner flask of desired width (4 cm). Once flat, the sample was further dried under a weight at 45° C. O/N. A foam of the PEG/chitin suspension was then produced on top of the backing films by dampening a backing film to adhere to the bottom of a beaker, then pouring PEG/chitin suspension (11.6 g) over the top. Finally, the resulting sample was then frozen at −4° C. O/N and lyophilised to give the desired backing film-foam haemostat patch (thickness ˜0.7 cm, width 4 cm). An image of the patch is shown at FIG. 1.

Outcome Measures

Parameters measured included blood pressure, heart rate, blood loss volume, and time to haemostasis (using protocols described in Padhye et al. (2015)). “Primary haemostasis” was said to be gained if haemostasis was achieved during the operation and up to 1 hour post-operation with MAP>55 mmHg. “Time to haemostasis” was taken in seconds from the time the aneurysm clip was removed to the time all instruments were removed from the nasal cavity in the absence of bleeding. “Blood loss” was measured in millilitres (mL) from suction canisters and calculated from the weight of soiled drapes in grams (g). A “secondary bleed” was deemed to have occurred if bleeding was noted from the operative site in the form of a neck hematoma or rapid exsanguination from the time the neck wound had been closed until the 3-month end point. “Pseudoaneurysm” was deemed to have occurred if features were noted on MRA scan or rupture of pseudoaneurysm occurred resulting in sheep death.

### Results

Statistical analysis was performed using SPSS. One-way ANOVA was performed utilising Kruskal-Wallis test with uncorrected Dunn's test to perform multiple comparisons of the haemostat agents relative to each other.

Short Term Outcomes

Haemorrhage control was achieved in all 18 sheep. Mean time to haemostasis was 124 seconds (95% Cl 70.9-177) in the Tachosil® patch group, 79.8 seconds in the Evicel®/SNoW™ group (95% Cl 69-90.6) and 67 seconds (95% Cl 52.3-81.8) in the β-chitin patch group. This was compared with historical AnastoClip vessel clip closures (LeMaitre Vascular, Inc.) and muscle patch controls (eg as described in Padhye et al., 2014, and Valentine et al., supra) using identical methodology: mean time to haemostasis for the vessel closures was 249.1 seconds (95% Cl 112.9-385.3) and the mean time for the muscle patch was 850.3 seconds (95% Cl 593.1-1108). The overall effect of the use of a haemostat agent was significant (P=0.0001). In particular, there was a significantly reduced time to haemostasis between Evicel®/SNoW™ vs. AnastoClip (P=0.007), Evicel®/SNoW™ vs. muscle patch (P=0.001), Tachosil® patch vs. muscle patch (P=0.027), β-chitin patch vs. Tachosil® patch (P=0.004), β-chitin patch vs. AnastoClip® (P=0.0003), and β-chitin patch vs. muscle patch (P=0.0001). The differences between the Tachosil® patch and the use of Evicel®/SNoW™ (P=0.264), the Tachosil® patch and AnastoClip (P=0.142), AnastoClip and the muscle patch (P=0.232), and the β-chitin and Evicel®/SNoW™ (P=0.384) were not significant.

Mean blood loss was 281 ml (95% Cl 106-456 ml) in the Tachosil® patch group, 150 ml (95% Cl 105-196 ml) in the Evicel®/SNoW™ group and 121 ml (95% Cl 97-146 ml) in the β-chitin patch group. In comparison, the mean blood loss was 146 ml (95% Cl 17-298 ml) for the AnastoClip vessel closure and 928 ml (95% Cl 406-1449 ml) for the muscle patch. The overall effect of the use of a haemostat agent on volume of blood loss was significant (P=0.005). Particularly, the difference in mean blood loss was significantly different in experiments using a muscle patch vs. AnastoClip (P=0.0008), muscle patch vs. Evicel®/SNoW™ (P=0.031), muscle patch vs. β-chitin patch (P=0.006) and AnastoClip vs. Tachosil® patch (P=0.010). The differences between the muscle patch vs. Tachosil® patch (P=0.212), AnastoClip vs. Evicel®/SNoW™ (P=0.181), AnastoClip vs. β-chitin patch (P=0.561), Tachosil® patch vs. Evicel®/SNoW™ (0.264), Tachosil® patch vs. β-chitin patch (P=0.071), and Evicel®/SNoW™ vs. β-chitin patch (P=0.490) were not significant.

Haemodynamic parameters did not vary significantly between the groups with mean arterial pressure (MAP) of 67.7 mmHg at the beginning of the procedure in the Tachosil® patch group, 69.8 mmHg in the Evicel®/SNoW™ group and 70 mmHg in the β-chitin patch group. MAP at the end of the procedure was 64.3 mmHg in the Tachosil® group, 62.5 mmHg in the Evicel®/SNoW™ group, 66 mmHg in the β-chitin patch group. The change in MAP was not significant for any group.

Long-Term Outcomes

One sheep in the Tachosil® patch group died of acute neck swelling on post-operative day 11 and autopsy revealed a large clot compressing the neck vessels. The carotid artery was removed and a pseudoaneurysm was discovered on histological examination with a defect in the muscular layer of the artery with disorganised collagen fibres overlying this and a fresh blood clot. Among the animals that received a β-chitin patch, two underwent humane killing on day 11 and day 13 post-operatively for infection not responsive to antibiotics. They underwent formal angiography prior to this which demonstrated no significant abnormalities of the carotid. The remainder of the sheep survived to 3 months and MRA of the neck vessels of these animals did not show any pseudoaneurysm development. Among the controls, one sheep in the AnastoClip group had an unruptured pseudoaneurysm and another sheep in the muscle patch group suffered a ruptured pseudoaneurysm. Histological examination of the carotid arteries did not demonstrate any adverse inflammatory response to any of the patches used.

### Discussion

Definitive haemostasis was achieved in all cases in this study. Further, there was a significant improvement in the time to haemostasis and blood loss for all three haemostat agent types compared to muscle patch controls. Moreover, blood loss was significantly reduced in all groups when compared with muscle patch controls. The β-chitin patch (a haemostat agent in accordance with the present disclosure) showed considerable promise with short term outcome (ie time to haemostasis, and blood loss) performance being as least as good as the commercially available Evicel®/SNoW™ product, and clearly outperforming the Tachosil® sealant patch on at least the time to haemostasis measure. As such, this (3-chitin haemostat agent represents a promising “ready-to-apply” patch for haemorrhage control in cases such as carotid injury. It appears to be safe, efficacious and capable of providing long term haemostasis out to three months or more with no significantly increased risk of pseudoaneurysm formation over muscle patch or commercially available vessel clip closures.

### Example 2

### Methods and Materials

Preparation ofβ-chitin Haemostat Agent (Patch)

Circular β-chitin patches were prepared to the following specifications using preparation methods substantially as described in Example 1:

| Specification A: | β-chitin bilayer patch (with PEG) | |
|---|---|---|
| | Dimensions: | 20 mm diameter |
| | First layer: | β-chitin/PEG (20% w/w) |
| | | foam; 4.55 mm thick |
| | Second (backing) layer: | β-chitin; 0.03 mm thick |
| Specification B | β-chitin bilayer patch (without PEG) | |
| (comparator): | Dimensions: | 20 mm diameter |
| | First layer: | β-chitin foam; |
| | | 4.55 mm thick |
| | Second (backing) layer: | β-chitin; 0.03 mm thick |
| Specification C | Single layer β-chitin patch (with PEG) | |
| (comparator): | Dimensions: | 20 mm diameter |
| | First layer: | β-chitin/PEG (20% w/w) |
| | | foam; 4.55 mm thick |

Gamma Irradiation and Assessment of Irradiated Patches

Patches according to Specification A were subjected to gamma irradiation (25 kGY) in accordance with standard methodologies for sterilisation of medical products. The irradiated patches were assessed for any changes to β-chitin fibre length (eg to observe whether any fibre breakdown was caused) by SEM microscopy.

Endotoxin Analysis

Gamma-irradiated patches according to Specification A were assessed for the presence of endotoxins according to standard methodologies (ie the Limulus amebocyte lysate (LAL) method). Briefly, 4X patches were pooled together in a sterile (pyrogen free) plastic container and then 40 ml LAL reagent water (10 ml per patch) set at 37° C. was added. The patches were allowed to soak in this water for 15 minutes at 37° C. and then left to cool down at room temperature (18° to 22° C.) for 45 minutes (ie total soaking time was 1 hour). Extract water was then decanted into another pyrogen free container and tested using a Kinetic-Chromogenic LAL method.

Patch Strength (“Burst”) Testing

Burst strength of β-chitin patches according to Specification A was assessed in comparison to a patch according to the above comparator Specification C. The Specification A patch comprises a backing layer of β-chitin/PEG, whereas the comparator patch has no backing layer. The patches were subjected to a probe (diameter 2.8 mm) using a TA+ texture analyser (Stable Micro Systems Ltd, Surrey, United Kingdom) at 1 mm/s enabling measurement of burst pressure (ie where the probe burst through the patch). The burst pressure was measured and averaged from tests of three patches of both types.

### Results

SEM microscopy of β-chitin patches before and after gamma irradiation showed that the gamma irradiation caused no significant change (eg breakdown), if any, to the fibre length or appearance of the β-chitin. In both cases, the fibres were very long; often more than 100 μm in length. A high magnification (×15000) SEM image of a gamma irradiated patch is shown at FIG. 2.

Gamma irradiated patches were also assessed for endotoxin content. Using a standard Kinetic-Chromogenic LAL method, performed by an independent laboratory, extract water showed a reading of 0.11 endotoxin units (EU)/ml which corresponds to 1.1 EU per patch. It is considered that this level would be acceptable for most applications of the patch. However, it is expected that improved (ie lowered) EU levels could be achieved by, for example, employing stricter physical containment (PC) protocols during patch manufacture.

β-chitin patches, with and without a β-chitin backing layer, were subjected to physical testing by measuring burst pressure with a 2.8 diameter probe. This showed that the backing layer greatly enhanced the strength of the patch; that is, with the backing layer present, the average burst pressure measured was 2500 kPa, whereas for single layer patches (ie with no backing layer), the average burst strength was just 150 kPa.

### Discussion

In this study, it was found that the fibrous β-chitin haemostat agents (patches) according to the present disclosure could readily withstand gamma irradiation for sterilisation and presented acceptable EU levels to permit their use in most clinical applications without the need for further steps to prevent or remove endotoxins. In addition, the presence of the second (backing) layer greatly enhanced the physical strength of the patches. The presence of a biocompatible polymer such as PEG in the first layer (eg a layer of β-chitin/PEG (20% w/w) foam) may provide the first layer with improved durability. However, surprisingly, the presence of PEG in the first layer was also observed to add a level of softness to the second (backing) layer. This was found to prevent the layers from separating (eg delaminating) during the preparation of the patches, especially upon drying (eg lyophilisation) (cf the image provided in FIG. 3 of a delaminated patch according to comparator Specification B). It is anticipated that this softness in the second (backing) layer will also be advantageous in applications where the patch requires folding (eg for keyhole surgery).

### Example 3

### Methods and Materials

Preparation of Various β-chitin Haemostat Agents (Patch)

Various square-shaped (1 cm×1 cm) β-chitin patches were prepared using preparation methods substantially as described in Example 1 (Table 1 below). Unless specifically stated, the first layer of the patches was about 0.07 cm thick, and the second layer was about 0.03-0.05 mm thick. In patches including calcium (Ca), this was introduced as calcium acetate (nb. in Table 1, “5% Ca” refers to 5% w/w of calcium acetate based on the amount of β-chitin present; similarly “10% Ca” refers to 10% w/w of calcium acetate based on the amount of β-chitin present, etc).

TABLE 1

| Patch name | First layer/Second layer | Comments |
|---|---|---|
| Chitin | β-chitin/β-chitin | |
| Chi/Thick | β-chitin/β-chitin | First layer was about |
| | | 0.14 cm thick |
| Chi/PP | β-chitin + 20% sodium | |
| | polyphosphate/β-chitin | |
| Chi/F127 | β-chitin + 20% Pluronic ® | |
| | F127 detergent/β-chitin | |
| Chi/PEG | β-chitin + 20% PEG/β-chitin | |
| Chi/5% Ca | β-chitin + 5% Ca/β-chitin | |
| Chi/10% Ca | β-chitin + 10% Ca/β-chitin | |
| Chi/20% Ca | β-chitin + 20% Ca/β-chitin | |

Platelet Aggregation Assay

Platelets were isolated and reconstituted to 2.5 mM CaCl₂ and 1.0 mM MgCl₂. 1.2 mL of platelet suspension was added to each patch and the sample was incubated for 60 minutes at 37° C. using a water bath. Following incubation, the patches were dip-rinsed twice in PBS (phosphate buffer solution) to remove all unattached platelets. The sample was then placed in PBS containing 0.9% Triton-X100 and further incubated for another 60 minutes at 37° C. For negative control (NC) and positive control (PC), 1.2 mL of platelet isolates were placed in PBS containing 0.9% and 20% Triton-X100, respectively, and incubated for 60 minutes at 37° C. The Triton-X100 caused lysis of all platelets attached to the patch, releasing lactate dehydrogenase (LDH) enzymes into the sample. The LDH levels within the sample was analysed using an LDH assay kit (Promega Corporation, Alexandria, NSW, Australia), as per manufacturer's instructions.

Thrombin Generation Assay

The patches were immersed in 1 mL of heparinised whole blood. Following a 10-minute incubation at 37° C., 20 μL of 0.633M of sodium citrate was added to the sample to stop thrombin generation. The sample was then centrifuged at 200×g for 10 minutes, after which the plasma was collected and the thrombin level was measured using Thrombin-Anti-thrombin Assay kit (Abcam Inc, Cambridge, Mass., United States of America), as per the manufacturer's instructions.

Cytotoxicity Assay

The PC-12 cell line was used in this assay. This is a cell line of neural origin, derived from rat pheochromocytoma. The PC-12 cells were cultured in Dulbecco's Modified Eagle Medium (DMEM) with 4.5 g/L D-Glucose, L-Glutamine and 25 mM HEPES (Gibco, Mulgrave, VIC, Australia), supplemented with foetal bovine serum, L-glutamate and penicillin and streptomycin. 1×10⁴ PC-12 cells were seeded into each well of a 96-well plate and incubated overnight at 37° C. in a 5% CO₂ in air atmosphere. Extracts of each of the chitin patches, Surgicel (Ethicon US LLC) and FloSeal haemostatic matrix (Baxter International Inc., Deerfield, Ill., United States of America) were formulated by immersing the haemostats in supplemented DMEM media at a concentration of 10 mg/mL. The whole sample was incubated overnight at 37° C. Following overnight incubation, the media was removed from the PC-12 cell culture and cells were treated with the chitin patch, Surgicel or FloSeal extract, culture media or 10% Triton X-100 in PBS. The cells were then further incubated at 37° C. for 24 hours and 48 hours. Following incubation, the culture media was removed and 100 μL of supplemented DMEM media comprising 10% alamar blue (Life Technologies, Mulgrave, VIC, Australia) was applied to the cell culture. Fluorescence was measured at 4-hours using 530 nm excitation and 590 nm emission. The raw data was analysed to evaluate cell viability.

Rat Femoral Arterial Bleed

Eighty male Wistar Albino rats (390-440 g), aged 8-10 weeks were randomly allocated to one of the following treatment groups: Chitin, Chi/F127, Chi/PEG, Chi/Ca, Chi/Thick, Surgicel® and FloSeal; with ten rats per group. Following allocation, the rats were weighed and anesthetized with 3% isoflurane. After induction, the animal's paw reflexes were checked to ensure complete induction. The right inguinal region was shaved and cleaned with ethanol prior to surgery. An inguinal incision was then made on the right leg, perpendicular to the inguinal canal. The proximal femoral artery was identified and was carefully dissected away from the surrounding femoral nerve, femoral vein and fascia. All instruments were moved away from the artery and a period of one minute was allowed to relieve any vasospasm. After the one-minute period, a standardized puncture was made on the anterior wall of the femoral artery using a 23-gauge needle. The artery was allowed to bleed freely for 5 seconds after which the treatment agent was applied to the site and it was compressed with the weight of a large, 250 g forceps. Pre-weighed gauze (G) was placed around the surgical site, to absorb the blood lost. An investigator blinded to the treatment decided when the bleeding had stopped and carefully recorded the bleeding time. The used gauze was re-weighed (W_{f}) and the total blood loss was calculated as, W_{f}−Wᵢ, and it was assumed that 1 g of blood is equivalent to mL of blood.

### Results

Platelet Aggregation Assay

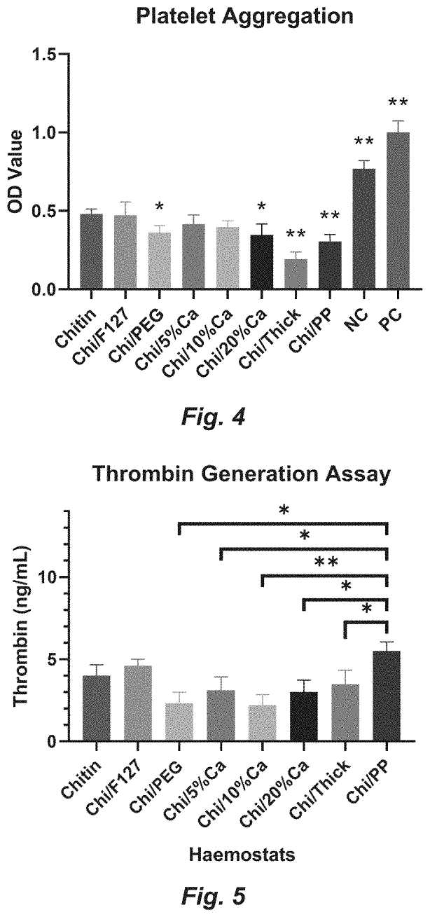

This assay investigated the aggregation of platelets to the β-chitin patches by measuring the relative quantity of platelets attached to the patch, as compared to a positive control. The chitin patch and Chi/F127 had the highest OD value compared to all other groups, indicating greater platelet aggregation ability (FIG. 4). Chi/PEG, Chi/20% Ca, Chi/Thick and Chi/PP, had significantly lower platelet aggregating index compared to chitin (p<0.05). Chi/Thick had the lowest platelet aggregation index and with the exception of Chi/PP (p=0.28), it was significantly lower than all other chitin patches (p<0.001). Further, increasing the concentration of calcium within the chitin patches resulted in a subsequent reduction in the platelet aggregation index.

Thrombin Generation Assay

This assay was aimed at investigating the ability of the β-chitin patches to induce the generation of thrombin. The results are shown in FIG. 5; the Chi/PP generated the maximum amount of thrombin, compared to all of the other patches, and this amount was significantly greater than Chi/Thick, Chi/PEG and the chitin patches including calcium (p<0.05).

Cytotoxicity Assay

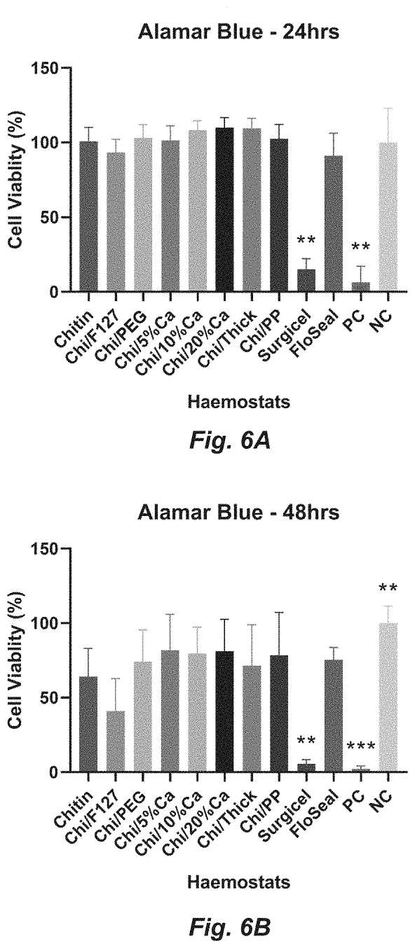

The potential for any cytotoxicity caused by the β-chitin patches was assessed. At 24 hours, the chitin patches and FloSeal had a cell viability of approximately 100% (FIGS. 6A and 6B), whereas, the cell viability with Surgicel® was approximately 15% and this was significantly lower than the chitin group (p<0.001). At 48 hours, there was a decrease in the cell viability for all of the haemostats and similar to 24 hour results, the cell viability with Surgicel was significantly lower than chitin (p<0.001) (FIGS. 6A and 6B). There were no significant differences between the chitin and FloSeal groups, at 24 or 48 hours; and it is considered that the β-chitin patches show only a very low and acceptable level of cytotoxicity.

Rat Femoral Arterial Bleed

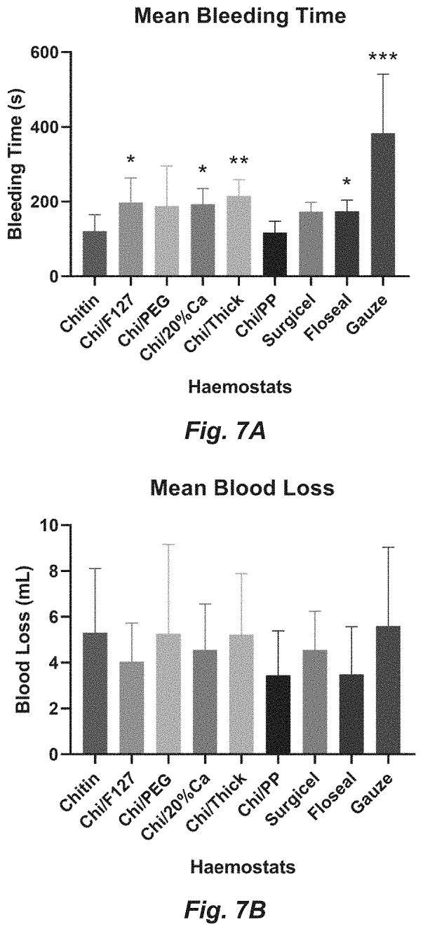

In this experiment, a comparison of the haemostatic effect was made between the various β-chitin patches, with gauze, Surgicel® and FloSeal using small vessel bleeds. The results are shown in FIGS. 7A and 7B. It was found that the β-chitin (Chitin) patch and Chi/PP had a mean bleeding time of 120.8 s and 117.3s, respectively, and this was approximately 60 seconds lower than most of the other haemostats tested (FIG. 7A). Both Chi/F127, Chi/20% Ca and Chi/Thick took approximately 180 seconds to achieve haemostasis, which was significantly longer than that required by the Chitin and Chi/PP patches (p<0.05). Also, Surgicel® and FloSeal took 172.7 seconds and 174.2 seconds on average to stop the bleeding, respectively and this was 50 seconds longer than the Chitin and Chi/PP patches (p >0.05). Gauze was the worst haemostat, requiring almost 400 seconds to stop the bleeding (FIG. 7A). There were no significant differences in the total blood loss between the groups (FIG. 7B).

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the haemostat agent of the present disclosure is not restricted in its use to the particular application described. Neither is the haemostat agent of the disclosure restricted in its preferred embodiment with regard to the particular elements and/or features described or depicted herein. It will be further appreciated that the haemostat agent is not limited to the embodiment or embodiments disclosed, but is capable of numerous rearrangements, modifications and substitutions without departing from the scope of the present disclosure as set forth and defined by the following claims.

### REFERENCES

Agrawal P et al., Int J Low Extrem Wounds 13(3):180-90 (2014).
Berker M et al., Pituitary 13(2):160-167 (2010).
Biswas D et al., J Laryngol Otol 123(6):692-694 (2009).
Gardner P A et al., Neurosurgery 73(2 Suppl Operative):ons 261-9; discussion ons 9-70 (2013).
Hickerson W L et al., Blood Coagul Fibrinolysis 22(1):19-23 (2011).
Kadokawa J-I et al., Carbohydrate Polymers 84(4):1408-12 (2011).
Kulling D et al., Endoscopy 30:41-42 (1998).
Matonick J P, and Hammond J. J Invest Surg 27(6):360-365 (2014).
Okamoto Y et al., Carbohydrate Polymers 53(3):337-342 (2003).
Padhye V et al., Int Forum Allergy Rhinol 4(8):651-657 (2014).
Padhye V V R, and Wormald, P J. Int Arch Otorhinolaryngol 18(S02):S173-S8 (2014).
Padhye V et al., Int Forum Allergy Rhinol 5(3):253-257 (2015).
Ray N H, Br Polym J 11(4):163-177, 1979
Travers R J et al., Int J Lab Hematol 37(01):31-35 (2015).Vaiman M. Otolaryngology-Head and Neck Surgery 126(4):388-391 (2002).
Vaiman M. Ann otot Rhinol Larvngol 112:410-414 (2003).
Valentine R et al., Int Forum Allergy Rhinol 1(2):118-122 (2011).
Valentine R, and Wormald P J. Skull Base 21(2):109-114 (2011).
Vaz-Guimaraes F et al., J Neurol Surg B Skull Base 76(4):296-302 (2015).
Whang H S et al., J. Macromol Sci, Part C: Polymer Reviews 45(4):309-23 (2005).
Thatte H S et al., The Journal of Trauma: Injury, Infection, and Critical Care 57(Supplement):S13-S21 (2004).

1. A haemostat agent in a form of a multi-layer patch comprising at least first and second layers, wherein said first layer comprises a composition comprising fibrous β-chitin and one or more biocompatible polymer, and said second layer comprises a film layer comprising fibrous β-chitin.

2. The agent of claim 1, wherein the first layer provides a haemostatic activity or effect and the second layer is a backing layer providing mechanical strength or reinforcement.

3. The agent of claim 2, wherein β-chitin is the only haemostatic carbohydrate polymer material present.

4. The agent of claim 2, wherein the composition of the first layer has a foamed structure or is otherwise comprised of a structure providing a random arrangement or network of voids and/or channels.

5. The agent of claim 4, wherein the composition of the first layer is freeze dried.

6. The agent of claim 2, wherein the β-chitin is obtained from a marine source.

7. The agent of claim 6, wherein the β-chitin is obtained from squid pens.

8. The agent of claim 2, wherein the β-chitin fibres of the first layer are coated, impregnated and/or suspended in one or more biocompatible polymer.

9. The agent of claim 8, wherein the one or more biocompatible polymer is present in the composition of the first layer in an amount of about 2 to about 50% w/w (based on the amount of β-chitin present).

10. The agent of claim 8, wherein the one or more biocompatible polymer is present in the composition of the first layer in an amount of about 20% w/w (based on the amount of β-chitin present).

11. The agent of claim 8, wherein the one or more biocompatible polymer is/are selected from the class of polyethylene glycol (PEG) polymers.

12. The agent of claim 11, wherein the one or more biocompatible polymer is a PEG compound with a molecular weight of about 1 kDa.

13. The agent of claim 2, wherein the one or more biocompatible polymer is sodium polyphosphate (PolyP), optionally provided as a functionalised surface on biocompatible and/or biodegradable nanoparticles.

14. A haemostat agent in a form of a multi-layer patch comprising at least first and second layers, wherein said first layer comprises a composition consisting of fibrous β-chitin and one or more biocompatible polymer, and said second layer comprises a film layer consisting of fibrous β-chitin.

15. The agent of claim 14, wherein the composition of the first layer is freeze dried.

16. The agent of claim 14, wherein the one or more biocompatible polymer is/are selected from the class of polyethylene glycol (PEG) polymers and is present in the composition of the first layer in an amount of about 20% w/w (based on the amount of β-chitin present).

17. The agent of claim 14, wherein the one or more biocompatible polymer is sodium polyphosphate (PolyP), optionally provided as a functionalised surface on biocompatible and/or biodegradable nanoparticles.

18. A haemostat agent in the form of a bi-layer patch consisting of first and second layers, wherein;
said first layer comprises a foamed composition comprising fibrous β-chitin and a biocompatible polymer selected from the class of polyethylene glycol (PEG) polymers, wherein said biocompatible polymer is present in an amount of about 20% w/w (based on the amount of β-chitin), and
said second layer is a film layer comprising fibrous β-chitin.

19. A haemostat agent in the form of a bi-layer patch consisting of first and second layers, wherein;
said first layer comprises a foamed composition comprising fibrous β-chitin and sodium polyphosphate, wherein said sodium polyphosphate is present in an amount of about 20% w/w (based on the amount of β-chitin), and
said second layer is a film layer comprising fibrous β-chitin.

20. A method of promoting haemostasis and/or wound healing in a human subject, said method comprising applying a haemostat agent according to claim 1 to a desired site such as a bleeding blood vessel, injury or other wound where bleeding management is desirable.

21. The method of claim 20, wherein the desired site is a site of a bleeding major blood vessel.

22. The method of claim 20, wherein the method is used for promoting haemostasis and/or wound healing in a carotid artery injury associated with endonasal surgery.

23. A method of producing a haemostat agent according to claim 1, comprising the steps of:
(i) forming a film of fibrous β-chitin,
(ii) forming a suspension of fibrous β-chitin and one or more biocompatible polymer;
(iii) applying the suspension of step (ii) to one side of the film of step (i) to produce a layered intermediate product, and
(iv) thereafter freeze drying the intermediate product such that a foamed composition of the β-chitin fibres and the biocompatible polymer is formed and provides a first layer that is bonded or laminated to the film which forms a second layer.
